(19) United States
(12) Patent Application Publication
(10) Pub. No.: US 2023/0049019 A1
(43) Pub. Date: Feb. 16, 2023

(54) INTRAORAL X-RAY SYSTEM

(71) Applicant: CARESTREAM DENTAL LLC, Atlanta, GA (US)

(72) Inventors: Jean-Marc INGLESE, Bussy-Saint-Georges (FR); David ROUDERGUES, Courbevoie (FR); Arnaud CAPRI, Croissy-Beaubourg (FR); Subramanyan KRISHNAMOORTHY, Penfield, NY (US); Vincent LOUSTAUNEAU, Fontenay sous Bois (FR)

(21) Appl. No.: 17/792,000

(22) PCT Filed: Dec. 29, 2020
(86) PCT No.: PCT/US2020/067244

§ 371 Date: Jul. 11, 2022

Related U.S. Application Data
(60) Provisional application No. 62/956,732, filed on Jan. 3, 2020.

(51) Int. Cl. A61B 6/14; A61B 6/00

(52) U.S. Cl. CPC A61B 6/145; A61B 6/527; A61B 6/547; A61B 6/4405; A61B 6/4411; A61B 6/4458; A61B 6/4476; A61B 6/025

(57) ABSTRACT

An intraoral x-ray system mountable to a dentist’s office wall including components movable to compensate for defects in the wall’s flatness or the wall not being sufficiently perpendicular to the floor. The system also includes monitoring and compensation capabilities to compensate for drift in the position of the system’s x-ray source or patient movement before and during x-ray imaging, thereby avoiding the need for the taking of additional x-ray images and exposing the patient unnecessarily to extra x-ray dose. Additionally, the system further includes a data/signal processing unit that allows the x-ray source to be precisely moved along a predetermined trajectory and allows the system to perform computed tomosynthesis examinations of a patient. In addition, the x-ray source is attachable/detachable from the system’s robotic arm, with the system compensating automatically for the change in weight at the robotic arm’s end due to removal of the x-ray source.

### FIELD OF THE INVENTION

The present invention relates, generally, to the field of x-ray systems for the health care industry and, more particularly, to intraoral x-ray systems used in the dental industry.

### BACKGROUND

Intraoral x-ray systems are generally used to provide two-dimensional (2D) images of a patient’s teeth. When a practitioner uses an intraoral x-ray system with a patient, an intraoral sensor is placed inside the patient’s mouth behind a tooth or teeth to be imaged and the system’s external x-ray source is brought near the patient’s face into the vicinity of the area to be imaged.

The x-ray sources of such intraoral x-ray systems are usually mounted to an articulated arm with the x-ray source being fixed at a first end of the articulated arm. A second end of the articulated arm may be fixed to a wall, on a dental chairside, or on a standalone base. If the second end of the articulated arm is fixed to a wall, the wall must be stable, flat, and perpendicular to the floor of the practitioner’s office. Because not all walls are stable, flat, and perpendicular to a floor, installation of the articulated arm to a wall can be challenging and time-consuming. If the articulated arm is attached to a dental chairside or standalone base, additional room is required around the dental chair which is often not available in many practitioner’s offices.

Regardless of where the articulated arm is affixed, the x-ray source is often heavy and, therefore, the articulated arm includes springs and cables to maintain the x-ray source stable while an x-ray image is taken of the patient’s tooth or teeth. Unfortunately, even with the springs and cables, drift instability may occur during x-ray imaging, causing blurring and other possible difficulties that affect image quality. And, if the wall is not sufficiently flat, stable, and perpendicular to the floor, the instability may be made worse.

Additionally, because the articulated arm is affixed to a wall, dental chairside, or standalone base, the x-ray source is generally limited to use only in a particular examination room, requiring the practitioner to equip his/her office with multiple x-ray sources in different examination rooms or requiring patients to be shuttled around the office between examination rooms in order for x-ray imaging. The practitioner’s investment in intraoral x-ray imaging systems could be reduced if the x-ray source were mobile. Also, with a mobile x-ray source, tomosynthesis examinations could be conducted in various examination rooms at minimal cost to the practitioner.

In addition, as previously mentioned, the practitioner moves the x-ray source near the patient’s face in order to perform the x-ray imaging, but it can sometimes be difficult for the practitioner to do so if the patient is moving. Further, if the patient moves slightly before or during x-ray imaging, the image quality will likely be adversely affected. If a satisfactory x-ray image is not obtained, the practitioner must take another x-ray, thereby increasing the x-ray dose to the patient.

Therefore, there is a need in the industry for an intraoral x-ray system that is simpler to install and use, that improves positioning of the x-ray source near the patient’s face, that produces quality x-ray images, and that solves these and other problems, difficulties and shortcomings of current systems.

### SUMMARY

Broadly described, the present invention comprises an intraoral x-ray system, including apparatuses and methods, for producing dental x-ray images. Advantageously, the intraoral x-ray system of the present invention makes installation simpler on a wall of a practitioner’s office because the movable components of the intraoral x-ray system can be moved to compensate for defects in the flatness of the wall or the wall not be sufficiently perpendicular to the floor. Also, due at least in part to its monitoring and compensation capabilities, the intraoral x-ray system can compensate for drift in the position of the x-ray source before and during x-ray imaging, thereby avoiding the need for the taking of additional x-ray images and exposing the patient unnecessarily to extra x-ray dose. The intraoral x-ray system can also, as described herein, compensate automatically for patient movements before and during x-ray imaging. And, because the x-ray source may be precisely moved under the control of the data/signal processing unit along a pre-determined trajectory, the intraoral x-ray system may be used to perform computed tomosynthesis examinations of a patient. Further, because the x-ray source and robotic arm are designed for the x-ray source to be attachable/detachable from the robotic arm and remainder of the intraoral x-ray system, no violent, unstable reaction occurs when the x-ray source is detached and removed from connection with the robotic arm. Instead, the data/signal processing unit detects the variation in weight at the second end of the robotic arm due to removal of the x-ray source and operates the robotic arm and other movable components of the intraoral x-ray system to automatically compensate for the variation in weight in a safe and predictable manner.

### BRIEF DESCRIPTION OF THE DRAWINGS

FIG. 1A displays a schematic representation of positioning of a patient and the patient’s vertical sagittal plane relative to the intraoral x-ray system in accordance with methods of example embodiments of the present invention.

FIG. 1B displays a schematic representation of positioning of a patient and the patient’s Frankfort plane relative to the intraoral x-ray system for maxillary imaging in accordance with methods of example embodiments of the present invention.

FIG. 1C displays a schematic representation of positioning of a patient and the patient’s occlusal plane relative to the intraoral x-ray system for mandibular imaging in accordance with methods of example embodiments of the present invention.

FIG. 2 displays a schematic representation of positioning of a patient’s tooth relative to the intraoral x-ray system using a positioning accessory of the intraoral x-ray system of example embodiments of the present invention in accordance with a paralleling method.

FIG. 3 displays a schematic representation of positioning of a patient’s tooth relative to the intraoral x-ray system using a positioning accessory of the intraoral x-ray system of example embodiments of the present invention in accordance with a bisecting method.

### DETAILED DESCRIPTION OF EXAMPLE EMBODIMENTS

Referring now to the drawings in which like elements or steps have the same reference numerals throughout the several views, the intraoral x-ray system 100 in a first example embodiment of the present invention comprises a robotic arm, an actuatable scissor arm, an x-ray source, and an intraoral x-ray detector. The robotic arm has an installation portion to be affixed to a wall, a chairside, or a mobile base and may have, in a second example embodiment, an optional extension arm portion connected to the installation portion. The actuatable scissor arm has a first end connected to the installation portion of the robotic arm or, in the second example embodiment, to the extension arm portion. In both the first and second example embodiments, the actuatable scissor arm is connected via a first rotatable, actuatable joint. The x-ray source is fixed at a second end of the actuatable scissor arm via a second rotatable, actuatable joint that is adapted to rotate the x-ray source along its vertical and horizontal axes. The x-ray source is optionally attachable/detachable from the second rotatable, actuatable joint. The rotatable, actuatable joints may be configured to rotate about a rotational axis or a rotational point.

In accordance with the first and second example embodiments, the intraoral x-ray system further comprises a driving unit and a position detector. The driving unit is configured to adjust the position of the x-ray source by operating at least one of the actuatable scissor arm, the first rotatable, actuatable joint, or the second rotatable, actuatable joint. The position detector is located near the x-ray source and is adapted to detect the position and orientation of the x-ray source. According to the first, second and other example embodiments, the position detector may comprise a gyroscope, and the intraoral x-ray system may further comprise an accelerometer near the x-ray source that is configured to detect acceleration of the x-ray source at least in one direction and a data/signal processing unit (such as, but not limited to, a microprocessor, digital signal processor, or other similar device) to detect drift of the x-ray source position and to compensate for the drift automatically in real time.

The x-ray source of the example embodiments of the present invention may comprise a conventional thermionic x-ray tube source or a cold-cathode x-ray source such as one that includes carbon nanotubes that reduce its weight and simplify the design of the robotic arm. The x-ray source may be controlled and powered through a connector on the robotic arm, and if the x-ray source is made attachable to/detachable from the robotic arm, the connector can support the attachment of a power module such as one that includes, without limitation, batteries or supercapacitors.

In the above-described or other example embodiments, the intraoral x-ray system further comprises a positioning accessory (such as, but not limited to, a position sensor or Easypos™ accessory) that is operable to indicate the position of the intraoral x-ray detector while inside the patient’s mouth. The position of the x-ray source can also be monitored relative to the position of the positioning accessory (and, hence, relative to the position of the intraoral x-ray detector) by the data/signal processing unit such that the data/signal processing unit may cause movement of the x-ray source (via movement of the rotatable arm and, possibly, other components) to compensate for patient movements before or during x-ray imaging. Additionally, the data/signal processing unit may determine the optimal position and displacement of the robotic arm and other actuatable components to pre-position the x-ray source relative to the patient and/or the positioning accessory prior to x-ray imaging.

According to still other example embodiments, the robotic arm may be operated and moved during x-ray computed tomography imaging to move the x-ray source along a desired trajectory and orientation. It should be understood and appreciated that the x-ray imaging system of the present invention may comprise other actuatable, movable and non-movable components connected to the other components of the system described herein to provide added flexibility for use of the system in various modalities and for various purposes.

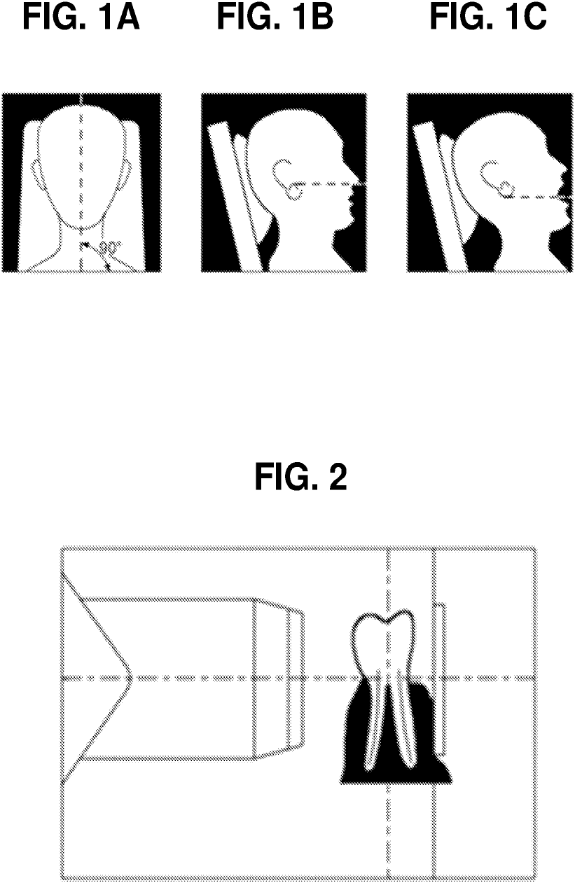

In accordance with methods of the example embodiments, the patient is positioned relative to x-ray intraoral x-ray system by aligning the patient’s head, as illustrated in FIG. 1A, with the vertical sagittal plane (indicated by the dashed line) such that the vertical sagittal plane bisects the patient’s head as viewed from left to right and is perpendicular to a horizontal plane. For upper maxillary imaging, the patient’s head is further positioned relative to the intraoral x-ray system, as illustrated in FIG. 1B, so that the Frankfort plane (nose-ear plane indicated by the dashed line) is horizontal. For lower mandibular imaging, the patient’s head is further positioned relative to the intraoral x-ray system, as illustrated in FIG. 1C, so that the occlusal plane (indicated by the dashed line) is horizontal.

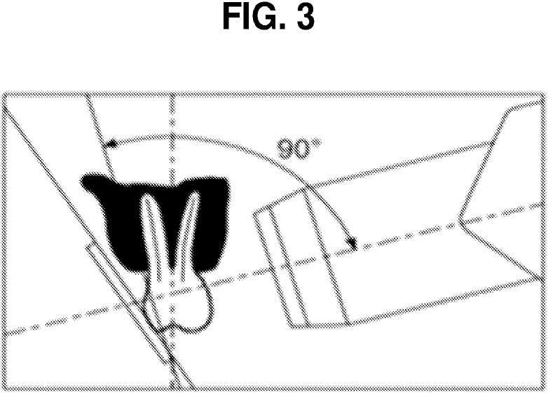

Next, the x-ray source is positioned relative to the patient’s head. The actuatable scissor arm allows accurate positioning of the x-ray source for any type of exposure or imaging modality. A beam-limiting (or collimator) device maintains a distance of at least 20 cm (8 in.) between the x-ray source’s focal spot and the patient’s skin, which allows use of either the paralleling or the bisecting methods. According to the paralleling method and as depicted in FIG. 2, the intraoral x-ray system uses the positioning accessory to align the x-ray beam emitted by the x-ray source and the intraoral x-ray detector. The collimator reduces the patient x-ray dose by limiting the surface exposure. According to the bisecting method and as depicted in FIG. 3, the intraoral x-ray system does not use a rectangular collimator. By not using a rectangular collimator, the risk of x-ray beam and intraoral x-ray detector misalignment is limited.

Advantageously, the intraoral x-ray system of the present invention makes installation simpler on a wall of a practitioner’s office because the movable components of the intraoral x-ray system can be moved to compensate for defects in the flatness of the wall or the wall not be sufficiently perpendicular to the floor. Also, due at least in part to its monitoring and compensation capabilities, the intraoral x-ray system can compensate for drift in the position of the x-ray source before and during x-ray imaging, thereby avoiding the need for the taking of additional x-ray images and exposing the patient unnecessarily to extra x-ray dose. The intraoral x-ray system can also, as described herein, compensate automatically for patient movements before and during x-ray imaging. And, because the x-ray source may be precisely moved under the control of the data/signal processing unit along a pre-determined trajectory, the intraoral x-ray system may be used to perform computed tomosynthesis examinations of a patient. Further, because the x-ray source and robotic arm are designed for the x-ray source to be attachable/detachable from the robotic arm and remainder of the intraoral x-ray system, no violent, unstable reaction occurs when the x-ray source is detached and removed from connection with the robotic arm. Instead, the data/signal processing unit detects the variation in weight at the second end of the robotic arm due to removal of the x-ray source and operates the robotic arm and other movable components of the intraoral x-ray system to automatically compensate for the variation in weight in a safe and predictable manner.

It should be understood and appreciated that while the present invention has been described in accordance with various example embodiments herein, the spirit and scope of the present invention shall not be limited by the example embodiments and includes other variations of the intraoral x-ray system.

What is claimed is:

1. An intraoral x-ray system, comprising:
a robotic arm having an installation portion for fixation to a wall of a dental practitioner’s examination room;
an actuatable scissor arm having a first end and a second end, wherein the first end is connected to the robotic arm via a first rotatable, actuatable joint; and
an x-ray source connected to the second end of the actuatable scissor arm via a second rotatable, actuatable joint.

2. The intraoral x-ray system of claim 1, wherein the installation portion is fixable to a dental chairside.

3. The intraoral x-ray system of claim 1, wherein the installation portion is fixable to a mobile base.

4. The intraoral x-ray system of claim 1, wherein the x-ray source is detachable from the second rotatable, actuatable joint.

5. The intraoral x-ray system of claim 1, wherein the x-ray source has vertical and horizontal axes, and the second rotatable, actuatable joint is adapted to rotate the x-ray source along the vertical and horizontal axes.

6. The intraoral x-ray system of claim 1, wherein the intraoral x-ray system further comprises an extension arm connected to the installation portion of the robotic arm.

7. The intraoral x-ray system of claim 1, wherein the intraoral x-ray system further comprises a driving unit configured to adjust the position of the x-ray source by operating at least one of the actuatable scissor arm, the first rotatable, actuatable joint, or the second rotatable, actuatable joint.

8. The intraoral x-ray system of claim 1, wherein the intraoral x-ray system further comprises a position detector located near the x-ray source and adapted to detect the position and orientation of the x-ray source.

9. The intraoral x-ray system of claim 8, wherein the position detector comprises a gyroscope.

10. The intraoral x-ray system of claim 1, wherein the intraoral x-ray system further comprises an accelerometer located near the x-ray source that is configured to detect acceleration of the x-ray source at least in one direction.

11. The intraoral x-ray system of claim 1, wherein the intraoral x-ray system further comprises a data/signal processing unit configured to detect drift of the x-ray source position and to compensate for the drift automatically in real time.

12. The intraoral x-ray system of claim 1, wherein the x-ray source comprises a cold-cathode x-ray source including carbon nanotubes.
